(19) United States
(12) Patent Application Publication
(10) Pub. No.: US 2026/0309714 A1
(43) Pub. Date: Oct. 8, 2026

(54) IMMUNOHISTOCHEMISTRY (IHC) PROTOCOLS AND METHODS FOR DIAGNOSING AND TREATING CANCER - CLAUDIN 18.2

(71) Applicant: Agilent Technologies, Inc., Santa Clara, CA (US)

(72) Inventors: Eunson JUNG, Santa Clara, CA (US); Sara DUESTERHOEFT, Santa Clara, CA (US); Donna KELL, Santa Clara, CA (US); Kurt MATHEWS, Santa Clara, CA (US); Edward MANNA, Santa Clara, CA (US)

(21) Appl. No.: 19/482,684

(22) PCT Filed: May 8, 2024
(86) PCT No.: PCT/US2024/028275

§ 371 Date: Nov. 7, 2025

Related U.S. Application Data
(60) Provisional application No. 63/464,795, filed on May 8, 2023.

(51) Int. Cl. G01N 33/575

(52) U.S. Cl. CPC G01N 33/5759; G01N 2333/705; G01N 2474/20

(57) ABSTRACT

In alternative embodiments, provided are immunohistochemistry (IHC) methods for determining and scoring reproducibly the extent of expression of the protein Claudin 18.2, in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring Claudin 18.2 expression and utilizing the score as a companion or complementary diagnostic or to treat or ameliorate cancer or a tumor.

### CROSS-REFERENCE TO RELATED APPLICATION

This patent application claims priority to U.S. Provisional Patent Application No. 63/464,795, filed on May 8, 2023, which is herein incorporated by reference in its entirety.

### TECHNICAL FIELD

This invention generally relates to cancer treatments, companion or complementary diagnostics and immunohistochemical methods. In alternative embodiments, provided herein are immunohistochemistry (“IHC”) methods for determining and scoring reproducibly the extent of expression of the protein Claudin 18.2 (also known as “CLDN18.2”), in a tissue sample. In alternative embodiments, provided are methods for diagnosing, treating or ameliorating or assessing the risk of recurrence for a cancer or a tumor using an IHC method as provided herein. In alternative embodiments, provided are kits comprising components and instructions for practicing methods as provided herein. The present application describes methods for scoring Claudin 18.2 expression and utilizing the score as a companion or complementary diagnostic or to treat or ameliorate cancer or a tumor.

### BACKGROUND

Cancer is the second leading cause of mortality worldwide, and the global burden is expected to rise to 29 million new cancer cases by 2040. Tight junction proteins are frequently dysfunctional or modified in several types of cancer cells, and their controlled paracellular permeation and polarity are lost in tumor cells. Tight junction proteins modulate cytoskeletal elements and signaling molecules bound to these proteins, leading to the loss of regulated cell migration and proliferation. Claudin 18.2 (UniProt ID: P56856-2; SEQ ID NO: 1), a 261 amino acid isoform of Claudin 18, is a member of the tight junction protein family. It is a highly selective marker protein expressed only in differentiated gastric mucosal membrane epithelial cells, with very limited expression in other normal healthy tissues, and no expression in undifferentiated gastric stem cells. There is evidence that abnormal Claudin 18.2 expression throughout the manifestation and growth of several primary and metastatic malignant tumors, such as gastric cancer (GC), gastroesophageal junction (GEJ) cancer, esophageal cancer, stomach cancer, gastric cancer metastases, colorectal cancer, colitis associated colorectal adenocarcinoma, colon cancer, non-squamous non-small cell lung cancer (nsNSCLC), non-small cell lung cancer (NSCLC), adenocarcinoma, cholangiocarcinoma, biliary intraepithelial neoplasia, pancreatic cancer, pancreatic adenocarcinoma, pancreatic cancer metastases, pancreaticobiliary adenocarcinoma, genitourinary tract cancer, biliary carcinoma, and biliary tract cancer. With its distinct pattern of expression, Claudin 18.2 has emerged as a unique molecule for targeted therapy for several cancers, particularly in gastric cancer (“GC”), which is currently the third most common and deadliest cancer globally, with poor prognosis, a high rate of recurrence and metastasis.

### SUMMARY

While it is known that Claudin 18.2 plays a key role in carcinogenesis, methods for scoring Claudin 18.2 expression and using the score as a companion or complementary diagnostic or to treat or ameliorate cancer or a tumor are still widely needed. Accordingly, the present disclosure addresses various needs in the art by providing methods for scoring Claudin 18.2 expression and utilizing the score as a companion or complementary diagnostic or to treat or ameliorate cancer or a tumor.

In a first general aspect, the disclosure provides an immunohistochemistry (IHC) method for determining and scoring the extent of cellular membrane expression of Claudin 18.2 in a tissue sample. In various aspects, such a method comprises: (a) staining a tissue sample with an antibody which specifically binds to Claudin 18.2; (b) determining a total number of viable neoplastic or cancer cells having Claudin 18.2 cellular membrane staining, and determining a total number of staining and non-staining viable neoplastic or cancer cells in at least a portion of the tissue sample,
wherein a neoplastic or cancer cell is counted as positively stained with anti-Claudin 18.2 antibody if there is Claudin 18.2 cellular membrane staining at any intensity above a defined threshold; and (c) determining a Claudin 18.2 score (%),
wherein the Claudin 18.2 score (%) is the number of Claudin 18.2 staining viable neoplastic or cancer cells found in the tissue sample divided by the total number of staining and non-staining viable neoplastic or cancer cells, multiplied by 100.

In some aspects, such a method further comprises excluding cells having pseudo-luminal, pseudo-apical, or secretion-type staining, wherein only neoplastic or cancer cells that exhibit membrane staining are considered positive for Claudin 18.2.

In some aspects, the tissue sample comprises at least about 100 cells.

In some aspects, a neoplastic or cancer cell counted as positive excludes staining of normal or non-neoplastic structures, staining nonviable neoplastic or cancer cells, necrotic cells, cellular debris, stromal staining, or edge artifact staining on a periphery of the tissue sample.

In some aspects, a defined intensity threshold comprises a positive intensity of cell staining.

In some aspects, the Claudin 18.2 score comprises the number of Claudin 18.2 viable neoplastic or cancer cells staining at a 1+, 2+ or 3+ positive staining intensity divided by the total number of staining and non-staining viable neoplastic or cancer cells, multiplied by 100.

In some aspects, a Claudin 18.2 score of about 50% or greater indicates a positive or negative interpretation of the tissue sample.

In some aspects, the Claudin 18.2 positivity score is about 5%, 10%, 20%, 30%, 40%, 50% or greater, about 60% or greater, about 80% or greater, or about 90% or greater.

In some aspects, a Claudin 18.2 score of about 5% or greater indicates a positive interpretation of the tissue sample.

In some aspects, a Claudin 18.2 score of about 5% or greater comprises the number of Claudin 18.2 viable neoplastic or cancer cells staining at 1+, 2+, or 3+ positive staining intensity; or a Claudin 18.2 score of about 20% or greater comprises the number of Claudin 18.2 viable neoplastic or cancer cells staining at a 1+, 2+, or 3+ positive staining intensity.

In some aspects, a section or portion of the tissue sample is prepared on a slide, a microscope slide, or equivalent, and the section or portion of the tissue sample is stained on the slide.

In some aspects, the antibody comprises a monoclonal mouse anti-Claudin 18.2 antibody or a monoclonal rabbit anti-Claudin 18.2 antibody.

In some aspects, the monoclonal rabbit anti-Claudin 18.2 antibody comprises monoclonal rabbit anti-Claudin 18.2 clone EPR19202, or an antibody having a substantially similar affinity for Claudin 18.2 as a clone EPR19202 antibody.

In some aspects, the tissue sample comprises a formalin-fixed, paraffin-embedded (FFPE) specimen.

In some aspects, the FFPE specimen comprises a cancer specimen stained on an automated IHC platform.

In some aspects, the section of the tissue sample is prepared by a protocol comprising fixation in about 10% neutral buffered formalin for a time period of about 6 hours to about 72 hours. In some aspects, the time period is from about 12 hours to about 60 hours, or from about 24 hours to about 48 hours.

In some aspects, the tumor or cancer is gastric cancer, a stomach cancer, a gastric cancer metastases, a gastroesophageal junction (GEJ) cancer, an esophageal cancer, a colorectal cancer, colitis associated colorectal adenocarcinoma, a colon cancer, a non-squamous non-small cell lung cancer (nsNSCLC), a non-small cell lung cancer (NSCLC), an adenocarcinoma, Cholangiocarcinoma, biliary intraepithelial neoplasia, Pancreatic cancer, pancreatic adenocarcinoma, pancreatic cancer metastases, pancreaticobiliary adenocarcinoma, Genitourinary tract cancer, biliary carcinoma, and biliary tract cancer.

In some aspects, the tissue sample is derived from a needle biopsy sample, a fine-needle aspirate, a cytology specimen, or a bone decalcification.

In some aspects, positive staining is determined using a bright-field light microscope, a microscope objective, a computer monitor and imaging software, or a combination thereof. In some aspects, the imaging software comprises whole slide imaging software.

In a second general aspect, the disclosure provides a method for treating or ameliorating a tumor or a cancer in a patient, comprising determining a Claudin 18.2 interpretive status in a tissue sample from the patient using a method of any of various embodiments for determining and scoring the extent of cellular membrane expression of Claudin 18.2 in a tissue sample herein, and treating the patient with a cancer therapeutic to which the patient is likely to respond favorably based on the Claudin 18.2 interpretive status.

In some aspects, the tissue sample has a Claudin 18.2 score of about 20% or greater of neoplastic or cancer cells having Claudin 18.2 cellular membrane staining at an intensity of 2+ or greater.

In some aspects, the tumor or cancer is a gastric cancer, a stomach cancer, a gastric cancer metastases, a gastroesophageal junction (GEJ) cancer, an esophageal cancer, a colorectal cancer, colitis associated colorectal adenocarcinoma, a colon cancer, a non-squamous non-small cell lung cancer (nsNSCLC), a non-small cell lung cancer (NSCLC), an adenocarcinoma, Cholangiocarcinoma, biliary intraepithelial neoplasia, Pancreatic cancer, pancreatic adenocarcinoma, pancreatic cancer metastases, pancreaticobiliary adenocarcinoma, Genitourinary tract cancer, biliary carcinoma, and biliary tract cancer.

In some aspects, the cancer therapeutic comprises administration to the patient an anti-cancer drug or an anti-cancer therapy.

In some aspects, the anti-cancer therapy comprises an antibody drug conjugate, a small molecule therapy, an immunotherapy, a monoclonal antibody therapy, an adoptive cell therapy, a T-cell receptor therapy, or a chimeric antigen receptor (CAR) T-cell therapy.

In a third general aspect, the disclosure provides a method for assessing the extent of Claudin 18.2 expression in a tissue sample from an individual comprising: contacting the tissue sample or a portion thereof comprising cancer or neoplastic cells with an antibody or a portion thereof which specifically binds to Claudin 18.2; determining a total number of viable neoplastic or cancer cells having Claudin 18.2 cellular membrane staining; determining a total number of staining and non-staining viable neoplastic or cancer cells; and determining a Claudin 18.2 score (%) by dividing the number of Claudin 18.2 staining viable neoplastic or cancer cells in the sample or portion thereof that are specifically bound by the antibody by the total number of staining and non-staining viable cancer or neoplastic cells and multiplying the result by 100, thereby obtaining the Claudin 18.2 score (%).

In a fourth general aspect, the disclosure provides a kit comprising an antibody which specifically binds to Claudin 18.2 and Claudin 18.2 scoring guidelines as set forth in various embodiments of methods for determining and scoring the extent of cellular membrane expression of Claudin 18.2 in a tissue sample herein.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates a representative image of Claudin 18.2 staining in gastric cancer using a Claudin 18.2 IHC (antibody clone) EPR19202 assay, as described herein.

FIG. 2 illustrates a representative image of positive staining examples: Convincing partial (red arrow, middle of) and complete linear (black arrow, or arow at middleright top) membrane staining are considered positive; a 20× image is shown here for detailed illustration, however scoring and calculation of % Claudin 18.2 positive membrane staining also can be measured using various objectives including 4×, 10×, 20× and 40×.

FIG. 3 illustrates an exemplary benign tissue staining example: staining of normal (non-neoplastic) tissue, as shown here, is excluded from Claudin 18.2 scoring protocols as provided herein.

FIGS. 4A-B illustrate images demonstrating that mucin can stain at high intensity (H&E staining FIG. 4A, and Claudin 18.2 staining FIG. 4B); this is not considered as non-specific background staining, and it is also excluded from Claudin 18.2 percent score using Claudin 18.2 scoring protocols as provided herein.

FIGS. 5A-F illustrate positive control tissue images: gastric cancer tissue at 10× (FIG. 5A, FIG. 5D), 20× (FIG. 5B, FIG. 5E), and 40× (FIG. 5C, FIG. 5F) with FIG. 5A, FIG. 5B, and FIG. 5C stained with negative control reagent and FIG. 5D, FIG. 5E, and FIG. 5F stained with anti-Claudin 18.2 antibody, demonstrating a range of weak (red arrow) to moderate (black arrow) intensity membrane staining using Claudin 18.2 scoring protocols as provided herein.

FIGS. 6A-D illustrate negative control tissue images: FIG. 6A: H&E stain of gastric cancer; FIG. 6B: a low magnification objective can be used to initially scan tissue and observe any Claudin 18.2 staining; FIG. 6C: a higher magnification of 10× or 20× can be used to confirm if there is any staining; and, FIG. 6D: a 20× or 40× objective can be used to visualize if there is any specific membrane staining at the 1+ intensity. In this example, there is no Claudin 18.2 staining and this specimen can serve as a good negative control tissue.

FIG. 7 illustrates an image of an H&E stain of a tissue specimen, which is evaluated first by H&E to assess tissue histology and preservation quality.

FIG. 8 illustrates a negative control reagent slide, this is used to evaluate non-specific staining and allow interpretation of a Claudin 18.2 stained slide.

The drawings set forth herein are illustrative of exemplary embodiments provided herein and are not meant to limit the scope of the invention as encompassed by the claims.

Figures are described in detail herein.

Like reference symbols in the various drawings indicate like elements.

### DETAILED DESCRIPTION

In alternative embodiments, provided herein are IHC methods for determining and scoring the extent of expression of the protein Claudin 18.2 (SEQ ID NO: 1; also known as CLDN18.2) in a tissue sample. In alternative embodiments, provided herein are scoring methods to assess Claudin 18.2 expression in neoplastic tissues, tumors or cancers such as a gastric cancer (“GC”), a stomach cancer, a gastroesophageal junction (“GEJ”) cancer, an esophageal cancer, a gastric cancer metastases, a colorectal cancer, a colon cancer, a colitis associated colorectal adenocarcinoma, a non-squamous non-small cell lung cancer (“nsNSCLC”), a non-small cell lung cancer (“NSCLC”), an adenocarcinoma, a cholangiocarcinoma, a biliary intraepithelial neoplasia, a pancreatic cancer, a pancreatic adenocarcinoma, a pancreatic cancer metastases, a pancreaticobiliary adenocarcinoma, a genitourinary tract cancer, a biliary carcinoma, and a biliary tract cancer.

Alternative embodiments of IHC methods herein provide robust, reliable, standardized, reproducible and harmonized methods for assessing the extent of cellular membrane expression of Claudin 18.2, thus creating greater between-laboratory and between-study comparability, and allowing earlier valid applications of Claudin 18.2 in clinical practice. Embodiments of IHC methods herein provide for high quality staining and reliable diagnostic assessment.

Current immunohistochemistry scoring methods for Claudin 18.2 measure the percentages of cells in a tissue sample that exhibit partial or whole membrane staining. The percentages are further divided into staining intensity bins. In order to determine a final Claudin 18.2 diagnostic score relative to the cutoff, only staining at 2+ and 3+ intensities are considered. Separation of membrane expression level scoring into multiple staining intensity bins adds complexity to the scoring process. The greater complexity of such scoring methods in turn adds to the time required to reach a diagnostic assessment. Pathologists can also vary in their individual determinations about the presence of or relative staining intensities of membrane staining patterns among the various cells in tissue samples, adding difficulty to reproducibility of scoring methods. Moreover, it can be confusing as to how to apply the scoring guidelines, since tumor morphology in some cancers is heterogeneous, including in gastric cancer. There remains a need in the art for simpler and more efficient, as well as robust, reproducible, and accurate scoring methods to assess Claudin 18.2 expression in tissue samples.

Embodiments herein are directed to immunohistochemistry methods for determining and scoring the extent of cellular membrane expression of Claudin 18.2 in a tissue sample. In various embodiments, the method comprises: (a) staining a tissue sample with an antibody which specifically binds to Claudin 18.2; (b) determining a total number of viable neoplastic or cancer cells having Claudin 18.2 cellular membrane staining, and determining a total number of staining and non-staining viable neoplastic or cancer cells in at least a portion of the tissue sample, wherein a neoplastic or cancer cell is counted as positively stained with anti-Claudin 18.2 antibody if there is Claudin 18.2 cellular membrane staining at any intensity above a defined threshold; and (c) determining a Claudin 18.2 score (%), wherein the Claudin 18.2 score (%) is the number of Claudin 18.2 staining viable neoplastic or cancer cells found in the tissue sample divided by the total number of staining and non-staining viable neoplastic or cancer cells, multiplied by 100.

In various embodiments, a method for assessing the extent of Claudin 18.2 expression in a tissue sample from an individual comprises: contacting the tissue sample or a portion thereof comprising cancer or neoplastic cells with an antibody or a portion thereof which specifically binds to Claudin 18.2; determining a total number of viable neoplastic or cancer cells having Claudin 18.2 cellular membrane staining; determining a total number of staining and non-staining viable neoplastic or cancer cells; and determining a Claudin 18.2 score (%) by dividing the number of Claudin 18.2 staining viable neoplastic or cancer cells in the sample or portion thereof that are specifically bound by the antibody by the total number of staining and non-staining viable cancer or neoplastic cells and multiplying the result by 100, thereby obtaining the Claudin 18.2 score (%). Such embodiments can provide not only robust, reliable, reproducible and accurate Claudin 18.2 scoring methods, but also scoring methods having greater simplicity and efficiency. Such embodiments can provide a considerable advantage for reliable and efficient diagnostic assessments.

In certain embodiments, the Claudin 18.2 cellular membrane staining comprises whole membrane staining, discontinuous membrane staining, partial membrane staining, complete membrane staining, linear membrane staining, or a combination thereof. In certain embodiments, the total number of viable neoplastic, tumor or cancer cells comprises multiple layers of cells, cells having signet ring morphology, or a combination thereof. Such embodiments can provide an advantage of including all observed patterns and levels of cellular membrane staining, and all types of viable neoplastic, tumor or cancer cells in the determination of a Claudin 18.2 positivity score. Such embodiments can provide a benefit of simplified IHC scoring methods for scoring cellular membrane expression of Claudin 18.2 in tissue samples.

In certain embodiments, the tissue sample comprises at least about 10⁰ cells. In certain embodiments, the tissue sample comprises at least about 200 cells. In certain embodiments, the tissue sample comprises at least about 500 cells.

In some embodiments, a neoplastic, tumor or cancer cell counted as positively stained excludes neoplastic, tumor or cancer cells having cytoplasmic staining, staining of normal or non-neoplastic structures, staining nonviable neoplastic, tumor or cancer cells, necrotic cells, cellular debris, stromal staining, or edge artifact staining on a periphery of the tissue sample. In certain embodiments, cytoplasmic staining comprises cytoplasmic staining at any (1+, 2+, 3+) positive staining intensity. Such embodiments can provide advantages of increasing the accuracy and reproducibility of Claudin 18.2 scoring methods by excluding stained cells having certain structures or characteristics from the Claudin 18.2 positivity score determination.

In certain embodiments, the defined threshold comprises a 1+ or greater positive staining intensity. In certain embodiments, the defined threshold comprises a 2+ or greater positive staining intensity. In certain embodiments, the defined threshold comprises a 2+ positive or a 3+ positive staining intensity. In certain embodiments, the Claudin 18.2 positivity score comprises the number of Claudin 18.2 viable neoplastic, tumor or cancer cells staining at a 2+ or greater positive staining intensity divided by the total number of staining and non-staining viable neoplastic, tumor or cancer cells, multiplied by 100. Such embodiments can provide a benefit of versatility in the staining intensity of the defined threshold.

In certain embodiments, a Claudin 18.2 positivity score of about 20% or greater indicates a diagnostic status of the tissue sample. In certain embodiments, a Claudin 18.2 positivity score of about 40% or greater indicates a diagnostic status of the tissue sample. In certain embodiments, the Claudin 18.2 positivity score is about 50% or greater, about 60% or greater, about 80% or greater, or about 90% or greater. In certain embodiments, a Claudin 18.2 positivity score of about 5% or greater indicates a diagnostic status of the tissue sample. In certain embodiments, a Claudin 18.2 positivity score of about 1% or greater indicates a diagnostic status of the tissue sample. In certain embodiments, a Claudin 18.2 positivity score of about 5% or greater comprises the number of Claudin 18.2 viable neoplastic, tumor or cancer cells staining at a 2+ or greater positive staining intensity; or a Claudin 18.2 positivity score of about 10% or greater comprises the number of Claudin 18.2 viable neoplastic, tumor or cancer cells staining at a 2+ or greater positive staining intensity. In certain embodiments, a Claudin 18.2 positivity score of about 20% or greater comprises the number of Claudin 18.2 viable neoplastic, tumor or cancer cells staining at a 2+ or greater positive staining intensity. Such embodiments can provide a benefit of accuracy in a diagnostic status determination related to Claudin 18.2 expression.

In alternative embodiments, provided are IHC methods comprising a simplified scoring system for membrane staining at certain intensity threshold, and provided is guidance for dealing with different morphological patterns of membrane staining. Inclusion of partial membrane staining of neoplastic or tumor cells in the Claudin 18.2 assessment when viability and neoplastic or tumor cell identity can be confirmed.

In alternative embodiments, methods and kits as provided herein are used for in vitro diagnostic uses. In alternative embodiments, the Claudin 18.2 IHC as provided herein is an IHC assay using an anti-Claudin 18.2 antibody such as a monoclonal rabbit anti-Claudin 18.2 clone EPR19202 (available from Abcam), or an antibody having a substantially similar affinity for Claudin 18.2 as a clone EPR19202 antibody, in the detection of a Claudin 18.2 protein in formalin-fixed, paraffin-embedded (FFPE) tissue samples such as lung carcinoma tissue sample. In alternative embodiments, the EnVision FLEX™ visualization system on Dako OMNIS™ is used.

In alternative embodiments, the Claudin 18.2 IHC as provided herein is used as an aid in identifying patients with a tumor or a cancer, for example, a gastric cancer (GC), gastroesophageal junction (GEJ) cancer, esophageal cancer, stomach cancer, gastric cancer metastases, colorectal cancer, colitis associated colorectal adenocarcinoma, colon cancer, non-squamous non-small cell lung cancer (nsNSCLC), non-small cell lung cancer (NSCLC), adenocarcinoma, cholangiocarcinoma, biliary intraepithelial neoplasia, pancreatic cancer, pancreatic adenocarcinoma, pancreatic cancer metastases, pancreaticobiliary adenocarcinoma, genitourinary tract cancer, biliary carcinoma, and biliary tract cancer.

In alternative embodiments, methods for assessing the extent of Claudin 18.2 expression comprise: contacting a sample or a portion thereof comprising cancer or tumor cells from an individual with an antibody or a portion thereof which specifically binds to Claudin 18.2; and determining a Claudin 18.2 positivity score (%) by dividing the number of Claudin 18.2 staining viable tumor or cancer cells in the sample or portion thereof specifically bound by the antibody with the total number of staining and non-staining viable cancer or tumor cells and multiplying the result by 100, thereby obtaining the Claudin 18.2 positivity score (%).

In certain embodiments, a section or portion of the tissue sample is prepared on a slide, a microscope slide, or equivalent, and the section or portion of the tissue sample is stained on the slide. In certain embodiments, the tissue sample comprises a formalin-fixed, paraffin-embedded (FFPE) specimen. In certain embodiments, the FFPE specimen comprises a cancer specimen stained on an automated IHC platform. In certain embodiments, the section of the tissue sample is prepared by a protocol comprising fixation in about 10% neutral buffered formalin for a time period of about 6 hours to about 72 hours. In certain embodiments, the tissue sample is derived from a needle biopsy sample, a fine-needle aspirate, a cytology specimen, or a bone decalcification.

In certain embodiments, the antibody comprises a monoclonal rabbit anti-Claudin 18.2 antibody or a monoclonal rabbit anti-Claudin 18.2 antibody. In some embodiments, the monoclonal rabbit anti-Claudin 18.2 antibody comprises monoclonal rabbit anti-Claudin 18.2 clone EPR19202, or an antibody having a substantially similar affinity for Claudin 18.2 as a clone EPR19202 antibody.

In certain embodiments, the tumor or cancer is a gastric cancer (GC), a gastroesophageal junction (GEJ) cancer, an esophageal cancer, a stomach cancer, a gastric cancer metastases, a colorectal cancer, a colitis associated colorectal adenocarcinoma, a colon cancer, a non-squamous non-small cell lung cancer (nsNSCLC), a non-small cell lung cancer (NSCLC), an adenocarcinoma, a cholangiocarcinoma, a biliary intraepithelial neoplasia, a pancreatic cancer, a pancreatic adenocarcinoma, a pancreatic cancer metastases, a pancreaticobiliary adenocarcinoma, a genitourinary tract cancer, a biliary carcinoma, and a biliary tract cancer.

In certain embodiments, the positive staining is determined using a bright-field light microscope, a microscope objective, a computer monitor and imaging software, or a combination thereof. In some aspects, the imaging software comprises whole slide imaging software.

### Embodiments of Methods for Diagnosing a Tumor or a Cancer

Embodiments herein provide methods for diagnosing a tumor or a cancer by determining if a tissue sample is positive for expression of Claudin 18.2. In various aspects, the method comprises: determining a Claudin 18.2 diagnostic status in a tissue sample by an IHC method for determining the extent of cellular membrane expression of Claudin 18.2 as provided herein, wherein a Claudin 18.2 positivity score of about 20% or greater of tumor cancer cells having Claudin 18.2 cellular membrane expression at an intensity of 2+ or greater is diagnostically positive. In certain embodiments, the tumor or cancer is a gastric cancer (GC), a gastroesophageal junction (GEJ) cancer, an esophageal cancer, a stomach cancer, a gastric cancer metastases, a colorectal cancer, a colitis associated colorectal adenocarcinoma, a colon cancer, a non-squamous non-small cell lung cancer (nsNSCLC), a non-small cell lung cancer (NSCLC), an adenocarcinoma, a cholangiocarcinoma, a biliary intraepithelial neoplasia, a pancreatic cancer, a pancreatic adenocarcinoma, a pancreatic cancer metastases, a pancreaticobiliary adenocarcinoma, a genitourinary tract cancer, a biliary carcinoma, and a biliary tract cancer.

### Embodiments of Methods for Treating Cancers and Tumors

Provided are methods for treating or ameliorating a tumor or a cancer in a patient, comprising determining and scoring the amount of Claudin 18.2 in a tissue sample from the patient using a method as provided herein, wherein if the tissue sample is determined or scored to have a high or a diagnostically positive Claudin 18.2 score, the patient is treated with a cancer therapeutic or an anti-cancer therapy to which the patient is likely to respond favorably. The tumor or cancer can be a gastric cancer (GC), a gastroesophageal junction (GEJ) cancer, an esophageal cancer, a stomach cancer, a gastric cancer metastases, a colorectal cancer, a colitis associated colorectal adenocarcinoma, a colon cancer, a non-squamous non-small cell lung cancer (nsNSCLC), a non-small cell lung cancer (NSCLC), an adenocarcinoma, a cholangiocarcinoma, a biliary intraepithelial neoplasia, a pancreatic cancer, a pancreatic adenocarcinoma, a pancreatic cancer metastases, a pancreaticobiliary adenocarcinoma, a genitourinary tract cancer, a biliary carcinoma, and a biliary tract cancer.

In certain embodiments, the cancer therapeutic comprises administration to the patient an anti-cancer drug or an anti-cancer therapy. In alternative embodiments, the anti-cancer treatment or therapy comprises: surgery such as cyberknife therapy and endoscopic mucosal resection; chemo-embolization; an ablation technique such as radiofrequency ablation (RFA), cryoablation and/or microwave ablation; and/or, radiation therapy such as stereotactic body radiation therapy.

In certain embodiments, the anti-cancer therapy comprises an antibody drug conjugate, a small molecule therapy, an immunotherapy, a monoclonal antibody therapy, an adoptive cell therapy, a T-cell receptor therapy, or a chimeric antigen receptor (“CAR”) T-cell therapy. In alternative embodiments, the anti-cancer treatment or therapy comprises: a HER2 therapeutic ado-trastuzumab emtansine (or KADCYLAR), pertuzumab (or PERJETA®), trastuzumab (or HERCEPTIN®), enhertu fam-trastuzumab deruxtecan-nxki (or ENHERTUR); tucatinib (or TUKYSAR); pembrolizumab (or KEYTRUDA™), nivolumab (or OPDIVO™), ipilimumab (or YERVOY™); ramucirumab (or CYRAMZA™); entrectinib (or ROZLYTREK™), Larotrectinib (or VITRAKVI®); or dostarlimab-gxly (or JEMPERLI).

In alternative embodiments, the anti-cancer treatment or therapy comprises use of an anti-cancer drug that can comprise an antibody that specifically or substantially binds to the cancer or tumor, wherein the antibody is conjugated to a cytotoxic agent, and optionally the cytotoxic agent comprises a radionuclide (optionally Yttrium-90, Iodine-131, Lutetium-177, Radium-223 chloride, strontium-89 chloride or samarium-153 EDTMP), diphtheria toxin, pseudomonas exotoxin A, denileukin diftitox, moxetumomab pasudotox, calicheamicin or N-acetyl-y-calicheamicin, emtansine or DM1, maytansine or derivatives thereof, SN-38 (or 7-Ethyl-10-hydroxycamptothecin), or auristatin or monomethyl auristatin E (MMAE).

### Immunohistochemistry

In alternative embodiments, immunohistochemistry methodologies and/or reagents used with methods and products of manufacture or kits as provided herein can include or comprise or comprise use of any IHC protocol, IHC armamentarium, device and/or image or data analysis system, for practicing IHC or IHC reagents known in the art, for example, as described in U.S. Pat. Nos. (USPNs) 10,634,590 (describing a slide holder assembly fixture for use in IHC); 10,565,479 (describing methods for identifying blurred areas in digital images of stained tissue); 10,564,076 (describing systems for analytical (or IHC) sample preparation); 10,551,395 (describing an automated histological staining system); 10,551,378 (describing a tissue staining method); 10,504,224 (describing a digital tissue image analysis system for IHC); 10,501,777 (describing simultaneous, multiplexed detection and quantification of protein expression in IHC); 10,488,340 (describing method for extracting an image of a target fluorophore in a biological material); 10,453,195 (describing methods of detecting tissue areas of interest using digital pathology imaging); 10,438,381 (describing devices, systems and methods for generating a digital image of a tissue section); 10,430,943 (describing methods and programs for automated nuclei area/number estimation for IHC image analysis); 10,416,176 (describing methods for processing specimens in an automated histological staining system); 10,393,633 (describing methods for processing and inhibiting the degradation of an IHC sample); 10,217,011 (describing handling of IHC slides); 10,209,165 (describing automated or semi-automated methods for assessing the quality of staining of a specimen containing cells); 10,126,216 (describing methods for fixing tissue samples for IHC); U.S. Pat. Nos. 9,423,322; 8,515,683 (describing methods and systems for automated detection of immunohistochemical (IHC) patterns); U.S. Pat. No. 10,816,443 (describing automated batch stainers for staining biological specimens on microscope slides); or, U.S. Patent Application Publication Nos: US 2019/0178867 A1 (describing detection of specific tissue objects within thin sections of tissue samples as imaged in a brightfield microscope without using a chromogenic stain that is specific to those tissue objects); US 2019/0156510 A1 (describing an image analysis method for analyzing an IHC tissue sample); US 2019/0293637 A1 (methods and systems for quantitative immunohistochemistry (IHC) of a target protein molecule); US 2019/0080450 A1 (describing an automated determination of the staining quality of an IHC stained biological sample); or, US 2020/0316589 A1 (describing a multi-well solid support vessel for the processing and testing of fixed biological materials).

In alternative embodiments, the antibodies, antigen binding fragments thereof, or monomeric or dimeric antigen binding proteins as provided herein (including for example synthetic or recombinant forms) used in IHC protocols, or kits, as provided herein are substantially purified or isolated or are in the form of an unpurified or partially purified culture supernatant.

In alternative embodiments, methods as provided herein can use or comprise reagents for detecting or visualizing an antibody-antigen interaction using any products or methods know in the art, for example, and IHC protocol or reagents.

In alternative embodiments, methods as provided herein comprise use of chromogenic immunohistochemistry (CIH), wherein a primary antibody (for example, a recombinant antibody (Ab), or antigen binding fragment thereof, or monomeric or dimeric antigen binding protein, as provided herein) or secondary antibody (for example, where the secondary antibody binds to the primary antibody, or the recombinant antibody (Ab), or antigen binding fragment thereof, or monomeric or dimeric antigen binding protein as provided herein,) is conjugated to an enzyme such as peroxidase, for example, an immunoperoxidase), for example, a horseradish peroxidase (HRP), that can catalyze a color-producing reaction. In alternative embodiments, a chromogenic moiety used in methods as provided herein is or comprises a coumarin; a rhodamine; 2,3,6,7-tetrahydro-11-oxo-1H,5H,11H-[1]benzopyrano[6,7,8-ij]quinolizine-10-carboxylic acid; 7-(diethylamino) coumarin-3-carboxylic acid; a coumarin derivative; a rhodamine derivative; a tetramethylrhodamine; a diarylrhodamine derivative; QSY 7; QSY 9; QSY 21; diazo chromophores; DABSYL; tartrazine; triarylmethane compounds; fast red; fast blue; fuchsin; Cascade Blue acetyl; Dapoxylsulfonic acid/carboxylic acid succinimidyl ester; DY-405; Alexa Fluor 405 succinimidyl ester; Cascade Yellow succinimidyl ester; pyridyloxazole succinimidyl ester (PyMPO); Pacific Blue succinimidyl ester; DY-415; 7-hydroxycoumarin-3-carboxylic acid succinimidyl ester; DYQ-425; 6-FAM phosphoramidite; Lucifer Yellow; iodoacetamide; Alexa Fluor 430 succinimidyl ester; Dabcyl succinimidyl ester; NBD chloride/fluoride; QSY 35 succinimidyl ester; DY-485XL; Cy2 succinimidyl ester; DY-490; Oregon Green 488 carboxylic acid succinimidyl ester; Alexa Fluor 488 succinimidyl ester; BODIPY 493/503 C3 succinimidyl ester; DY-480XL; BODIPY FL C3 succinimidyl ester; BODIPY FL C5 succinimidyl ester; BODIPY FL-X succinimidyl ester; DYQ-505; Oregon Green 514 carboxylic acid succinimidyl ester; DY-510XL; DY-481XL; 6-carboxy-4′,5′-dichloro-2′,7′-dimethoxyfluorescein succinimidyl ester (JOE); DY-520XL; DY-521XL; BODIPY R6G C3 succinimidyl ester; erythrosin isothiocyanate; 5-carboxy-2′,4′,5′,7′-tetrabromosulfonefluorescein succinimidyl ester; Alexa Fluor 532 succinimidyl ester; 6-carboxy-2′,4,4′,5′7,7′-hexachlorofluorescein succinimidyl ester (HEX); BODIPY 530/550 C3 succinimidyl ester; DY-530; BODIPY TMR-X succinimidyl ester; DY-555; DYQ-1; DY-556; Cy3 succinimidyl ester; DY-547; DY-549; DY-550; Alexa Fluor 555 succinimidyl ester; Alexa Fluor 546 succinimidyl ester; DY-548; BODIPY 558/568 C3 succinimidyl ester; Rhodamine red-X succinimidyl ester; QSY 7 succinimidyl ester; BODIPY 564/570 C3 succinimidyl ester; BODIPY 576/589 C3 succinimidyl ester; carboxy-X-rhodamine (ROX); succinimidyl ester; Alexa Fluor 568 succinimidyl ester; DY-590; BODIPY 581/591 C3 succinimidyl ester; DY-591; BODIPY TR-X succinimidyl ester; Alexa Fluor 594 succinimidyl ester; DY-594; carboxynaphthofluorescein succinimidyl ester; DY-605; DY-610; Alexa Fluor 610 succinimidyl ester; DY-615; BODIPY 630/650-X succinimidyl ester; erioglaucine; Alexa Fluor 633 succinimidyl ester; Alexa Fluor 635 succinimidyl ester; DY-634; DY-630; DY-631; DY-632; DY-633; DYQ-2; DY-636; BODIPY 650/665-X succinimidyl ester; DY-635; Cy5 succinimidyl ester; Alexa Fluor 647 succinimidyl ester; DY-647; DY-648; DY-650; DY-654; DY-652; DY-649; DY-651; DYQ-660; DYQ-661; Alexa Fluor 660 succinimidyl ester; Cy5.5 succinimidyl ester; DY-677; DY-675; DY-676; DY-678; Alexa Fluor 680 succinimidyl ester; DY-679; DY-680; DY-682; DY-681; DYQ-3; DYQ-700; Alexa Fluor 700 succinimidyl ester; DY-703; DY-701; DY-704; DY-700; DY-730; DY-731; DY-732; DY-734; DY-750; Cy7 succinimidyl ester; DY-749; DYQ-4; Cy7.5 succinimidyl ester; 7-diethylaminocoumarin-3-carboxylic acid; succinimidyl ester; Dabsyl sulfonyl chloride; fluorescein isothiocyanate (FITC) carboxy succinimidyl ester (DY-495); Rhodamine Green carboxylic acid succinimidyl ester (DY-505); eosin isothiocyanate (EITC); 6-carboxy-2′,4,7,7′-tetrachlorofluorescein succinimidyl ester (TET); carboxyrhodamine 6G succinimidyl ester; carboxytetramethylrhodamine succinimidyl ester (TMR, TAMRA) (DY-554); QSY 9 succinimidyl ester; sulforhodamine B sulfonyl chloride (DY-560); Texas Red (sulforhodamine 101); gallocyanine; Fast Green FCF; Malachite Green; or, a QSY 21 succinimidyl ester.

In alternative embodiments, methods as provided herein comprise use of immunofluorescence, where a primary or a secondary antibody is tagged to a fluorophore, such as fluorescein or fluorescein isothiocyanate (FITC), a triarylmethane dye such as rhodamine or rhodamine derivatives (for example, tetramethylrhodamine (TRITC), rhodamine 6G, rhodamine 123, rhodamine B, carboxytetramethylrhodamine (TAMRA), tetramethylrhodamine (TMR), sulforhodamine 101), aminomethylcoumarin acetate (AMCA), ALEXA™ or DYLIGHT™ fluors, or a fluorophore or dye as described in U.S. patent application no. US 2019/0018018 A1. 3,3′-Diaminobenzidine (DAB) also can be used.

In alternative embodiments, methods as provided herein comprise use of a direct method or one-step staining method where a primary antibody (for example, antibodies (Ab), or antigen binding fragments thereof, or monomeric or dimeric antigen binding proteins as provided herein (including for example synthetic or recombinant forms)) is labeled and reacts directly with an antigen, for example, in a tissue section. While this technique utilizes only one antibody and therefore is simple and rapid, the sensitivity may be lower due to little signal amplification.

In alternative embodiments, methods as provided herein comprise use of an indirect method where an unlabeled primary antibody (first layer) binds to a target antigen (for example, Claudin 18.2), for example, in a tissue or organ, and a labeled secondary antibody (second layer) then is reacted with the primary antibody. The secondary antibody can be against the isotype, for example, IgG, of the animal species in which the primary antibody is derived. This method can be more sensitive than direct detection strategies because of signal amplification due to the binding of several secondary antibodies to each primary antibody if the secondary antibody is conjugated to a detecting agent such as a fluorescent or enzyme reporter.

In alternative embodiments, further amplification is achieved if the secondary antibody is conjugated to several detecting molecules, for example, biotin molecules, which can recruit complexes of avidin-, streptavidin- or NEUTRAVIDIN™ protein-bound enzyme.

In alternative embodiments, the IHC is performed on tissue sections or tissue biopsies, for example, paraformaldehyde (PFA) fixed tissues or organs, or formalin-fixed paraffin-embedded tissues. In alternative embodiments, a tissue is sectioned or sliced or used whole. Before sectioning, the tissue sample can be embedded in a medium, for example, paraffin wax or cryomedia. Tissue sections can be sectioned or sliced on a variety of instruments, most commonly using a microtome, cryostat, or vibratome. Specimens can be sectioned or sliced at a range of about 3 μm to 5 μm. The sections or slices can be mounted on slides, dehydrated using alcohol washes of increasing concentrations (for example, 50%, 75%, 90%, 95%, 100%), and cleared using a detergent like xylene before being imaged under a microscope.

Depending on the method of fixation and tissue preservation, the sample may require additional steps to make a Claudin 18.2 epitope available for antibody binding, including deparaffinization and antigen retrieval. For formalin-fixed paraffin-embedded tissues, antigen-retrieval is often necessary, and can comprise pre-treating the sections with heat or proteases.

In alternative embodiments, the IHC is performed using an ENVISION DUOFLEX DOUBLESTAIN SYSTEM™ (EnVision DuoFLEX Doublestain System) (Agilent, San Jose, CA), which allows for staining of two or more markers on a single slide. In alternative embodiments, the IHC is performed using an EnVision FLEX HRP Magenta, High pH (Dako Omnis) system, and binding can be visualized by EnVision FLEX HRP Magenta Chromogen. In alternative embodiments, the IHC is performed using En Vision FLEX Mini Kit, High pH, which is a high-sensitivity visualization system intended for use in IHC together with Dako AUTOSTAINER™ instruments or the Dako OMNIS instruments; this dual link system detects primary mouse and rabbit antibodies and the reaction is visualized by 3,3′-Diaminobenzidine (DAB) chromogen (DAB forms a water-insoluble brown precipitate when oxidized, for example, by a peroxidase).

### Products of Manufacture and Kits

Provided are products of manufacture and kits for practicing methods as provided herein, including for example, monoclonal rabbit anti-Claudin 18.2 antibodies such as clone EPR19202 anti-Claudin 18.2 antibodies, and/or reagents for practicing IHC, including for example reagents as described herein, see Example 1, below; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition, or a staining intensity of 0, 1+, 2+, or 3+.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### Examples

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK).

Exemplary Claudin 18.2 staining protocols are described in Example 2, below.

### Example 1: Claudin 18.2 IHC

This example demonstrates use of Claudin 18.2 staining protocols as provided herein.

Provided here are exemplary scoring guidelines for evaluation of Claudin 18.2 expression in gastric cancer specimens that have been stained with the rabbit monoclonal Claudin 18.2 antibody, designated rabbit mAb clone EPR19202. This antibody is provided in Claudin 18.2 IHC EPR19202 assay for the identification of Claudin 18.2 expression in formalin-fixed, paraffin-embedded (FFPE) gastric cancer specimens using the Dako Omnis automated staining system.

Assessment of Claudin 18.2 IHC Staining Patterns in Gastric Cancer FFPE Tissues. The protocol-specific Pathology Report Form (PRF) is completed for each specimen evaluated (including “Not Evaluable” and “Indeterminate” as described below).

### Claudin 18.2 Scoring Summary: Definition of Positive Staining

Claudin 18.2 positive staining is defined as any partial or complete neoplastic or tumor cell plasma membrane staining at an intensity threshold. Membrane staining may be partial or complete.

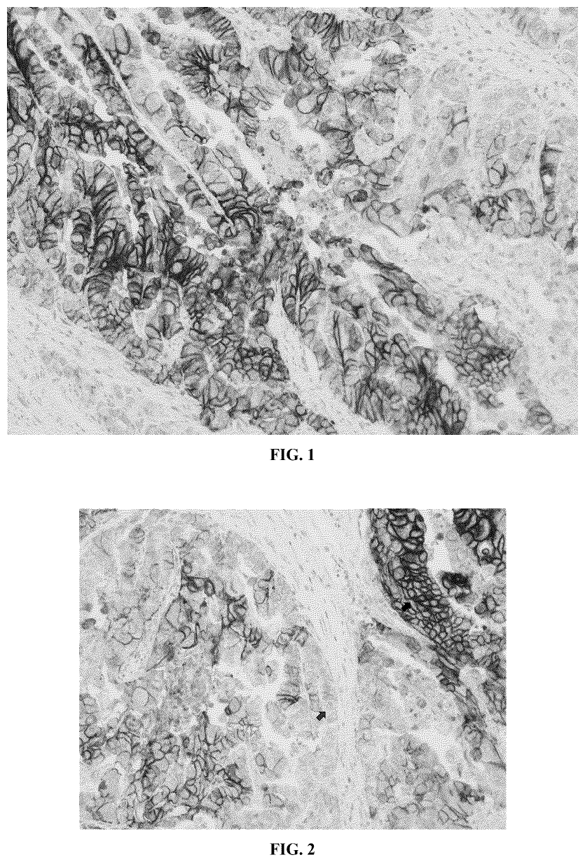

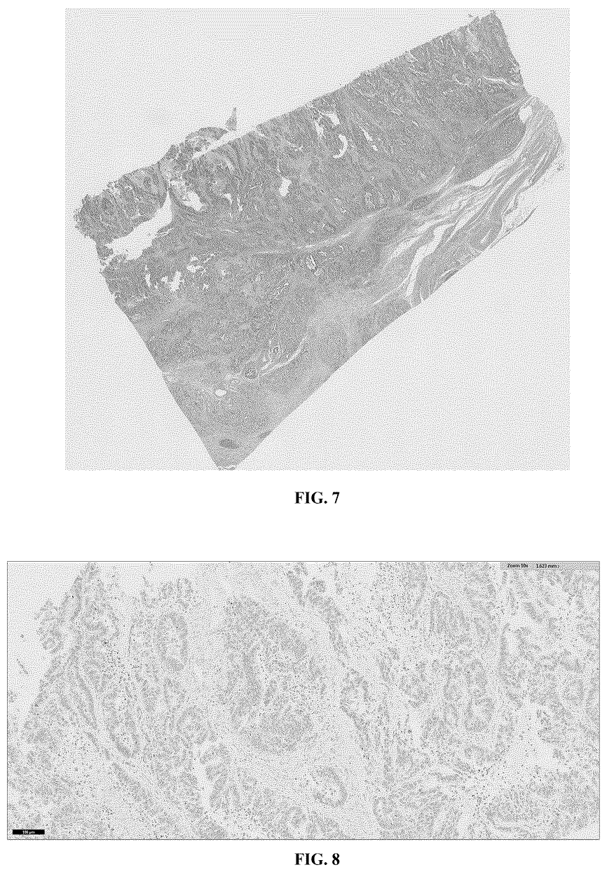

Representative staining examples are described below, see FIGS. 1 to 8.

Claudin 18.2 percent positive score is determined by the following formula:

$$\% CLDN 18.2 positive = 100 \times \frac{\# of tumor cells expressing CLDN 18.2 membrane stainin \geq X intensity}{Total \# of viable tumor cells present in the section}$$

A minimum of 100 evaluable and viable neoplastic or tumor cells must be present in the Claudin 18.2 stained slide to determine the percentage of stained cells.

Overall Claudin 18.2 diagnostic status is determined according to the following criteria:
The specimen is considered to be diagnostically positive if ≥X % of the neoplastic or tumor cells exhibit Claudin 18.2 positive staining at an intensity threshold, where X is the predetermined cut-off.
The specimen is considered to be diagnostically negative if <X % of the neoplastic or tumor cells exhibit Claudin 18.2 positive staining at an intensity threshold, where X is the predetermined cut-off.

### Claudin 18.2 Scoring Summary: Definition of Negative Staining

Negative staining is defined as absence of membrane staining.

Included in scoring:
Partial or complete membrane staining at any intensity or an intensity threshold is considered positive for Claudin 18.2 expression.
All viable neoplastic or tumor cells on a slide are evaluated for both intensity and frequency of positive staining.

Cell types and staining patterns to exclude from stain scoring:
Cytoplasmic staining
Staining of normal, non-neoplastic structures
Sloughed non-viable neoplastic or tumor cells
Necrotic cells and cellular debris
Stromal staining
Edge artifact staining on periphery of tissue specimen
In-situ carcinoma

### Scoring of Gastric Cancer

The staining intensity is scored using the following scale (Table 1):

TABLE 1
Staining Intensity Scale

| | | Staining intensity | Staining intensity |
|---|---|---|---|
| | | considered positive | considered positive |
| Intensity | Staining | for Claudin 18.2 | for Claudin 18.2 |
| Scale | Intensity | expression? | diagnosis? |
| 0 | No staining | No | No |
| 1+ | Weak staining | Yes | No |
| 2+ | Moderate | Yes | Yes |
| | staining | | |
| 3+ | Strong staining | Yes | Yes |

This assay is being evaluated for gastric cancer tissue samples. H&E stained slides should accompany every Claudin 18.2 stained sample to allow proper confirmation of the presence of tumor and histological diagnosis, assess tumor viability, and to allow distinctions from non-tumor portions of the slide which should be excluded from the assessment. H&E and NCR stained slides may also assist with identifying intrinsic pigments (due to intrinsic anthracosis, hemosiderin pigment, etc.).

Cells with Signet Ring Morphology: Cells with signet ring cell morphology can exhibit high intensity staining in the cytoplasm. Membrane staining in these cases may be difficult to differentiate due to the cytoplasmic staining intensity. For cells with signet ring cell morphology, the high intensity staining tumor cells are considered positive for membrane staining if partial or complete membrane staining is discernible from cytoplasmic staining.

High Cytoplasmic Staining Cells: Cytoplasmic staining without unequivocal membrane staining is not considered as positive staining. However, many gastric cases with membranous expression of Claudin 18.2 also exhibit cytoplasmic expression. Tumor cells are scored as positive for Claudin 18.2 when cells have discernible membrane staining from cytoplasmic staining.

### Non-Specific Background Staining:

No membrane staining on tumor cells should be observed on the negative control reagent (NCR) stained slides.

Non-specific staining on non-scorable tissue components is the following:
Serum
Plasma cells
Smooth muscle
Extracellular mucin
Necrotic debris
Endogenous or other pigment including but not excluded to hemosiderin, iron, melanin, surgical tattoo ink, tissue orienting ink, or endogenous peroxidase reactivity associated with red blood cells
Necrosis

Non-specific staining with the tumor region should not interfere with the interpretation of membrane staining on tumor cells.

### System Controls

Before examining the IHC stained tumor specimen(s), confirm that the IHC staining of control tissues is satisfactory.

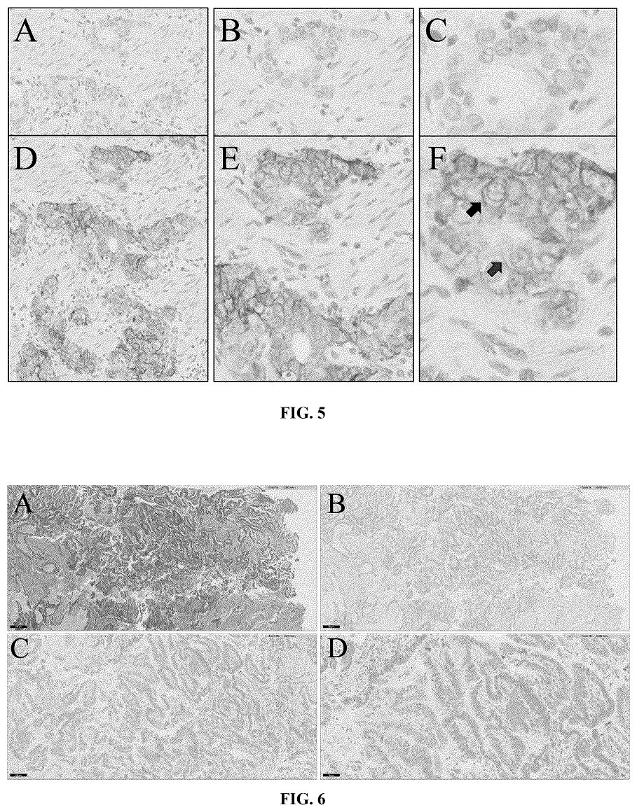

A positive control tissue and a negative control tissue are run in parallel with patient tissues. The NCR staining is run on control tissues in order to confirm the absence of non-specific staining for the control tissues within that staining procedure. See FIG. 5 for a representative image of a Positive Control Tissue and FIG. 6 for a representative image of a Negative Control Tissue.

### Control Tumor Tissues:

Control tumor tissues should be identified from pre-screened gastric cancer tissues. Controls should be fresh biopsy/surgical gastric cancer specimens fixed, processed and embedded as soon as possible in the same manner as the test specimen(s).

### Negative Control Reagent (NCR)

Negative Control Reagent contains a species matched antibody non-specific to Claudin 18.2. Control tissues stained with NCR must exhibit no specific membrane staining, and the presence of non-specific background staining on tumor cells should not interfere with the interpretation of membrane staining on tumor cells. If specific membrane staining occurs in the NCR stained tissues, then results are considered invalid, the checkbox for “Fail” should be recorded on the scoring form, and the staining should be repeated on the controls and test specimens.

### Positive Control Tissues

Positive control tissues are evaluated to ensure tissue preparation and satisfactory staining techniques. One positive control tissue is included in each staining procedure.
Tissues selected for use as the positive tissue controls should give moderately positive staining to detect subtle changes in assay sensitivity.
Specimens processed differently from the test sample(s) validate reagent performance only and do not verify tissue preparation.
Known positive tissue controls should only be utilized for monitoring the correct performance of processed tissues and test reagents, NOT as an aid in formulating a specific diagnosis of patient samples.

### Negative Control Tissues

Gastric cancer negative control tissues are evaluated to ensure tissue preparation, satisfactory staining techniques, verify the specificity of the primary antibody, and to provide an indication of non-specific staining.
Different cell types present in most tissue sections offer internal negative control sites (this should be verified by the user).
A suggested negative control tumor tissue for gastric cancer is one that shows no membrane staining in tumor cells.
Known negative tissue controls should only be utilized for monitoring the correct performance of processed tissues and test reagents, NOT as an aid in formulating a specific diagnosis of patient samples.

### Acceptance Criteria for Control Specimens

Based on the staining results of the control specimens, accept or reject the results of the test specimen according to the following criteria:
Accept the test specimen results if controls meet all requirements.
If control tissue slides do not meet requirements, then test specimen results should be considered invalid and the staining should be repeated, including tissue controls and tissue samples.
If Negative Control Reagent (NCR) staining results do not meet requirements, then test specimen results should be considered invalid and the staining should be repeated, including tissue controls and tissue samples.

Once proper staining of the control materials has been established, the staining is judged to be satisfactory for assessment of test samples.

Patient Tissue Slide Stained with NCR

Negative Control Reagent is used in place of the Claudin 18.2 specific primary antibody and aids in interpretation of specific staining at the antigen site.

Absence of membrane staining verifies the specific labeling of the target antigen by the Claudin 18.2 primary antibody on the patient specimen. Staining by the NCR must not show any membrane staining at any intensity.

### Slide Reading Order and Evaluation

For each staining run, slides should be examined in the order presented in the table below to determine the validity of the staining run and enable assessment of stained sample tissue.

| Specimens | Rationale | Requirements |
|---|---|---|
| 1. Positive | The positive control tissue | Controls should be |
| control tissue | stained with Claudin 18.2 | biopsy/surgical specimens, fixed, |
| stained with | primary antibody should be | processed and embedded as soon |
| Claudin 18.2 | examined first. Known positive | as possible in the same manner as |
| primary | control tissue should only be | the patient tissue(s). |
| antibody | utilized for monitoring the | Positive control tissue should be |
| (Lab- | correct performance of processed | included in each staining |
| supplied) | tissues and test reagents, NOT as | procedure. On-slide tissue |
| | an aid in formulating a specific | controls are recommended |
| | diagnosis of patient tissue. | Positive control tissue should be: |
| | | gastric cancer specimen |
| | | previously screened by the |
| | | laboratory and known to express |
| | | Claudin 18.2. Tumor cells should |
| | | exhibit membrane staining at |
| | | moderate intensity. |
| | | If the positive control tissue fails to |
| | | demonstrate appropriate positive |
| | | staining, then results of the test |
| | | specimen (patient tissue) should be |
| | | considered invalid. |
| 2. Positive | Negative Control Reagent may | Tissue stained with NCR must |
| control tissue | be used to stain the positive | exhibit no membrane staining. |
| stained with | control tissue specimen if | |
| Negative | needed for troubleshooting | |
| Control | purposes. | |
| Reagent | | |
| (NCR) | | |
| (Lab- | | |
| supplied) | | |
| 3. Negative | The negative control tissue | Controls should be |
| control tissue | stained with Claudin 18.2 | biopsy/surgical specimens, fixed, |
| stained with | primary antibody should be | processed and embedded as soon |
| Claudin 18.2 | examined next to verify the | as possible in the same manner as |
| primary | labeling specificity of the target | the patient tissue(s). |
| antibody | antigen by the primary | Negative control tissue should be |
| (Lab- | antibody. | included in each staining |
| supplied) | | procedure. On-slide tissue |
| | | controls are recommended. |
| | | Negative control tissue should be: |
| | | gastric cancer specimen |
| | | previously screened by the |
| | | laboratory and known to not |
| | | express Claudin 18.2 staining. |
| | | Tumor cells should exhibit no |
| | | membrane staining. |
| | | If the negative control tissues fail |
| | | to meet the above specifications, |
| | | the results of the patient tissue |
| | | should be considered invalid. |
| 4. Negative | Negative Control Reagent may | Tissue stained with NCR must |
| control tissue | be used to stain the negative | exhibit no membrane staining in |
| stained with | control tissue specimen if | tumor cells. |
| Negative | needed for troubleshooting | |
| Control | purposes. | |
| Reagent | | |
| (NCR) | | |
| (Lab- | | |
| supplied) | | |
| 5. Patient | A hematoxylin and eosin | The H&E and Claudin 18.2 |
| tissue stained | (H&E) stain of the patient | antibody stain should be |
| with H&E | tissue is evaluated first to | performed on serial sections from |
| | assess tissue histology and | the same paraffin block of the |
| | preservation quality. | specimen. A minimum of 100 |
| | | viable tumor cells must be present |
| | | for the specimen to be considered |
| | | adequate for Claudin 18.2 |
| | | evaluation. |
| | | Tissue specimens should be |
| | | undamaged, well-preserved, and |
| | | should confirm tumor indication. |
| 6. Patient | Examine patient tissue stained | Patient tissue stained with NCR |
| tissue stained | with the Negative Control | must exhibit no membrane |
| using the | Reagent. Negative Control | staining in tumor cells. |
| Negative | Reagent is used in place of the | If patient tissue stained with NCR |
| Control | primary antibody and aids in | fails to demonstrate appropriate |
| Reagent | interpretation of specific | staining, the corresponding patient |
| (NCR) | staining at the antigen site. | tissue stained with primary |
| | | antibody is considered non- |
| | | evaluable and the patient tissue |
| | | must be retested. |
| 7. Patient | Examine the patient tissue | All viable tumor cells on the entire |
| tissue stained | stained with the Claudin 18.2 | Claudin 18.2-stained slide must be |
| using the | antibody last to assess Claudin | evaluated for Claudin 18.2 and |
| Claudin 18.2 | 18.2 protein status. | included in the scoring |
| primary | | assessment. A minimum of 100 |
| antibody | | viable tumor cells must be present |
| | | for the specimen to be considered |
| | | adequate for Claudin 18.2 |
| | | evaluation. |
| | | Positive staining intensity should |
| | | be assessed within the context of |
| | | any non-specific staining |
| | | observed in the staining |
| | | procedure. NCR is recommended |
| | | for this assessment if non-specific |
| | | staining is observed. |
| | | As with any |
| | | immunohistochemical test, a |
| | | result showing no staining means |
| | | that the antigen was not detected, |
| | | not necessarily that the antigen |
| | | was absent in the cells/tissue |
| | | assayed. |
| | | Refer to Scoring Guidelines for |
| | | details on patient tissue |
| | | evaluation. |

### NCR Stained Specimen

The Negative Control Reagent (NCR) should be examined with a section of each patient specimen to evaluate non-specific staining and allow interpretation of specific staining.

### Acceptance Criteria:

Staining by NCR must not exhibit any specific membrane staining at any intensity.

### Claudin 18.2Stained Specimen

Carefully examine the entire tumor specimen and determine the percentage of Claudin 18.2 positive tumor cells at ≥2+ intensity:

$$\% CLDN 18.2 positive = 100 \times \frac{\# of tumor cells expressing CLDN 18.2 membrane stainin \geq X intensity}{Total \# of viable tumor cells present in the section}$$

The estimated number of total viable tumor cells in the entire tissue section will be used as the denominator for computation of the score.

The Claudin 18.2 diagnostic status (positive/negative) of gastric cancer specimens is determined by the percentage of viable tumor cells expressing Claudin 18.2 positive (≥2+ intensity) above an intensity threshold for membrane staining.
Tumor cells are positive for Claudin 18.2 expression if they exhibit either partial or complete circumferential membrane staining at any intensity.
Claudin 18.2 staining may appear as partial or complete membrane staining as illustrated in FIG. 2.
Review Claudin 18.2 expression patterns for important scoring details.

Conduct scoring of Claudin 18.2 positivity with the following resolution: in the range from zero to 10%, score positivity in 1% increments, except when in the range from zero to 1%, designate “<1%”. In the range from 10% to 100%, positivity may be scored in 5% increments, but smaller increments may be used if necessary.

Elements to Exclude from Scoring and from Evaluation of Non-Specific Background

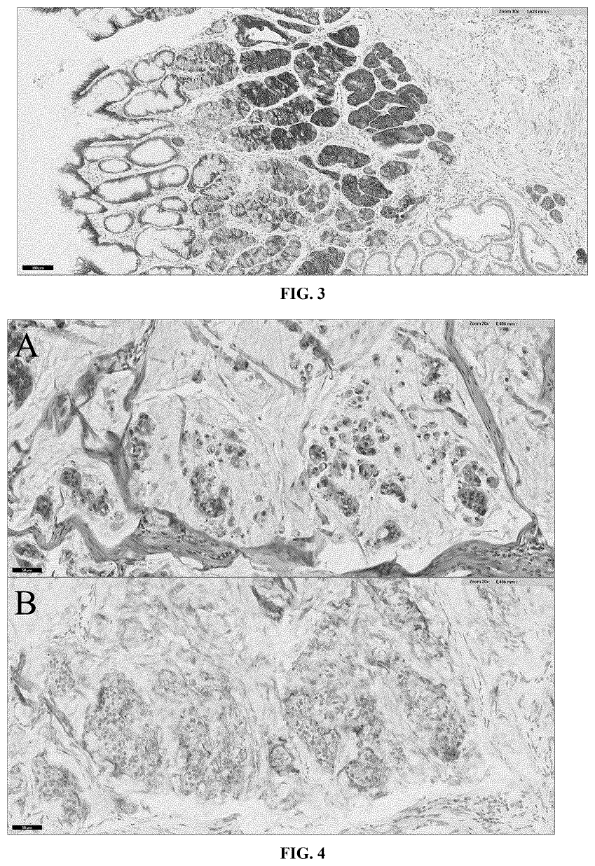

Additional tissue elements that may be present and may show apparently genuine Claudin 18.2 staining include:
Mucin (see FIG. 4)
Benign epithelial cells (see FIG. 3)

Staining of these areas are excluded from determining Claudin 18.2 expression status.

The Claudin 18.2 positivity status is determined based on pre-determined cut-off point of Claudin 18.2 positive staining at an intensity threshold and recorded.

The term “indeterminate” is utilized to denote a sample that has met the criteria for evaluation, but scoring of tumor cell membrane staining has been hampered for reasons attributable to the biology of the tumor tissue sample rather than because of improper sample preparation or handling.

Staining may also occur in certain regions of a tissue specimen such as the stroma, necrotic areas, and edge artifact in peripheral regions of the tissue. Stromal staining may occur in specimens exhibiting highly positive Claudin 18.2 expression. Staining of these tissue elements are excluded from determining Claudin 18.2 expression status and from the evaluation of non-specific background staining.

### Alternative Embodiments

In alternative embodiments, provided are IHC methods for determining and scoring the extent of cellular membrane expression of or Claudin 18.2 in a tissue sample, comprising:
staining a tissue sample with an antibody which specifically binds to Claudin 18.2;
determining a total number of viable neoplastic, tumor or cancer cells having Claudin 18.2 cellular membrane staining, and determining a total number of staining and non-staining viable neoplastic, tumor or cancer cells in at least a portion of the tissue sample,
wherein a neoplastic, tumor or cancer cell is counted as positively stained with anti-Claudin 18.2 antibody if there is Claudin 18.2 cellular membrane staining at any intensity above a defined threshold; and
determining a Claudin 18.2 positivity score (%),
wherein the Claudin 18.2 positivity score (%) is the number of Claudin 18.2 staining viable neoplastic, tumor or cancer cells found in the tissue sample divided by the total number of staining and non-staining viable neoplastic, tumor or cancer cells, multiplied by 100.

In alternative embodiments of IHC methods as provided herein:
the Claudin 18.2 cellular membrane staining comprises whole membrane staining, discontinuous membrane staining, partial membrane staining, complete (circumferential) membrane staining, linear membrane staining, or a combination thereof;
the total number of viable neoplastic, tumor or cancer cells comprises all viable neoplastic, tumor or cancer cells in the specimen, including multiple layers of cells, cells having signet ring morphology, or a combination thereof;
the tissue sample comprises at least about 100 viable neoplastic, tumor or cancer cells;
a neoplastic, tumor or cancer cell counted as positively stained excludes neoplastic, tumor or cancer cells having exclusively cytoplasmic staining of normal or non-neoplastic structures, staining nonviable neoplastic, tumor or cancer cells, necrotic cells, cellular debris, stromal staining, or edge artifact staining on a periphery of the tissue sample;
the Claudin 18.2 positivity score comprises the number of Claudin 18.2 viable neoplastic, tumor or cancer cells staining at an intensity staining threshold divided by the total number of staining and non-staining viable neoplastic, tumor or cancer cells, multiplied by 100; or, a Claudin 18.2 positivity score of about 40% or greater indicates a diagnostic status of the tissue sample; or, the Claudin 18.2 positivity score is about 50% or greater, about 60% or greater, about 80% or greater, or about 90% or greater;
a Claudin 18.2 positivity score of about 5% or greater indicates a diagnostic status of the tissue sample; or, a Claudin 18.2 positivity score of about 5% or greater comprises the number of Claudin 18.2 viable neoplastic, tumor or cancer cells staining at a 2+ or greater positive staining intensity; or a Claudin 18.2 positivity score of about 10% or greater comprises the number of Claudin 18.2 viable neoplastic, tumor or cancer cells staining at a 2+ or greater positive staining intensity; or a Claudin 18.2 positivity score of about 20% or greater comprises the number of Claudin 18.2 viable neoplastic, tumor or neoplastic or cancer cells having Claudin 18.2 cellular membrane staining at an intensity of 2+ or greater;
a section or portion of the tissue sample is prepared on a slide, a microscope slide, or equivalent, and the section or portion of the tissue sample is stained on the slide;
the antibody comprises a monoclonal rabbit anti-Claudin 18.2 antibody or a monoclonal rabbit anti-Claudin 18.2 antibody; or, the rabbit anti-Claudin 18.2 antibody comprises monoclonal rabbit anti-Claudin 18.2 clone EPR19202, or an antibody having a substantially similar affinity for Claudin 18.2 as a clone EPR19202 antibody;
the tissue sample comprises a formalin-fixed, paraffin-embedded (FFPE) specimen; or, the FFPE specimen comprises a cancer specimen stained on an automated IHC platform;
the section of the tissue sample is prepared by a protocol comprising fixation in about 10% neutral buffered formalin for a time period of about 6 hours to about 72 hours;
the tumor or cancer is a gastric cancer, a stomach cancer, a gastric cancer metastases, a gastroesophageal junction (GEJ) cancer, an esophageal cancer, a colorectal cancer, colitis associated colorectal adenocarcinoma, a colon cancer, a non-squamous non-small cell lung cancer (nsNSCLC), a non-small cell lung cancer (NSCLC), an adenocarcinoma, Cholangiocarcinoma, biliary intraepithelial neoplasia, Pancreatic cancer, pancreatic adenocarcinoma, pancreatic cancer metastases, pancreaticobiliary adenocarcinoma, Genitourinary tract cancer, biliary carcinoma, and biliary tract cancer;
the tissue sample is derived from a surgical resection or biopsy, needle biopsy sample, a fine-needle aspirate, a cytology specimen, or a bone decalcification; and/or
a positive staining is determined using a bright-field light microscope, a microscope objective, a computer monitor and imaging software, or a combination thereof; and optionally, the imaging software comprises whole slide imaging software.

The references DOI 10.21037/tcr-19-1876 and DOI 10.1080/21688370.2021.1967080 are related to Claudin 18.2 and gastric cancer.

SEQUENCE LISTING
SEQ ID NO.: 1
MAVTACQGLGFVVSLIGIAGIIAATCMDQWSTQDLYNNPVTAV
FNYQGLWRSCVRESSGFTECRGYFTLLGLPAMLQAVRALMIVG
IVLGAIGLLVSIFALKCIRIGSMEDSAKANMTLTSGIMFIVSG
LCAIAGVSVFANMLVTNFWMSTANMYTGMGGMVQTVQTRYTFG
AALFVGWVAGGLTLIGGVMMCIACRGLAPEETNYKAVSYHASG
HSVAYKPGGFKASTGFGSNTKNKKIYDGGARTEDEVQSYPSKH
DYV

1. An immunohistochemistry (IHC) method for determining and scoring the extent of cellular membrane expression of Claudin 18.2 in a tissue sample, comprising:
(a) staining a tissue sample with an antibody which specifically binds to Claudin 18.2;
(b) determining a total number of viable neoplastic or cancer cells having Claudin 18.2 cellular membrane staining, and determining a total number of staining and non-staining viable neoplastic or cancer cells in at least a portion of the tissue sample,
wherein a neoplastic or cancer cell is counted as positively stained with anti-Claudin 18.2 antibody if there is Claudin 18.2 cellular membrane staining at any intensity above a defined threshold; and
(c) determining a Claudin 18.2 score (%),
wherein the Claudin 18.2 score (%) is the number of Claudin 18.2 staining viable neoplastic or cancer cells found in the tissue sample divided by the total number of staining and non-staining viable neoplastic or cancer cells, multiplied by 100.

2. The method of claim 1, further comprising excluding cells having pseudo-luminal, pseudo-apical, or secretion-type staining, wherein only neoplastic or cancer cells that exhibit membrane staining are considered positive for Claudin 18.2.

3. The method of claim 1, wherein the tissue sample comprises at least about 100 cells.

4. The method of claim 1, wherein a neoplastic or cancer cell counted as positive excludes staining of normal or non-neoplastic structures, staining nonviable neoplastic or cancer cells, necrotic cells, cellular debris, stromal staining, or edge artifact staining on a periphery of the tissue sample.

5. The method of claim 1, wherein a defined intensity threshold comprises a positive intensity of cell staining.

6. The method of claim 5, wherein the Claudin 18.2 score comprises the number of Claudin 18.2 viable neoplastic or cancer cells staining at a 1+, 2+ or 3+ positive staining intensity divided by the total number of staining and non-staining viable neoplastic or cancer cells, multiplied by 100.

7. The method of claim 1, wherein a Claudin 18.2 score of about 5% or greater indicates a positive or negative interpretation of the tissue sample.

8. The method of claim 7, wherein the Claudin 18.2 positivity score is about 10%, 20%, 30%, 40%, 50% or greater, about 60% or greater, about 80% or greater, or about 90% or greater.

9. The method of claim 1, wherein a Claudin 18.2 score of about 5% or greater indicates a positive interpretation of the tissue sample.

10. The method of claim 9, wherein a Claudin 18.2 score of about 5% or greater comprises the number of Claudin 18.2 viable neoplastic or cancer cells staining at 1+, 2+, or 3+ positive staining intensity; or a Claudin 18.2 score of about 20% or greater comprises the number of Claudin 18.2 viable neoplastic or cancer cells staining at a 1+, 2+, or 3+ positive staining intensity.

11. The method of claim 1,
wherein a section or portion of the tissue sample is prepared on a slide, a microscope slide, or equivalent, and the section or portion of the tissue sample is stained on the slide, or
wherein the antibody comprises a monoclonal mouse anti-Claudin 18.2 antibody or a monoclonal rabbit anti-Claudin 18.2 antibody, optionally wherein the monoclonal rabbit anti-Claudin 18.2 antibody comprises monoclonal rabbit anti-Claudin 18.2 clone EPR19202, or an antibody having a substantially similar affinity for Claudin 18.2 as a clone EPR19202 antibody.

12. (canceled)

13. (canceled)

14. The method of claim 1, wherein the tissue sample comprises a formalin-fixed, paraffin-embedded (FFPE) specimen.

15. The method of claim 14,
wherein the FFPE specimen comprises a cancer specimen stained on an automated IHC platform,
wherein the section of the tissue sample is prepared by a protocol comprising fixation in about 10% neutral buffered formalin for a time period of about 6 hours to about 72 hours, or
wherein the time period is from about 12 hours to about 60 hours, or from about 24 hours to about 48 hours

16. (canceled)

17. (canceled)

18. The method of claim 1, wherein the tumor or cancer is gastric cancer, a stomach cancer, a gastric cancer metastases, a gastroesophageal junction (GEJ) cancer, an esophageal cancer, a colorectal cancer, colitis associated colorectal adenocarcinoma, a colon cancer, a non-squamous non-small cell lung cancer (nsNSCLC), a non-small cell lung cancer (NSCLC), an adenocarcinoma, Cholangiocarcinoma, biliary intraepithelial neoplasia, Pancreatic cancer, pancreatic adenocarcinoma, pancreatic cancer metastases, pancreaticobiliary adenocarcinoma, Genitourinary tract cancer, biliary carcinoma, or biliary tract cancer.

19. The method of claim 1,
wherein the tissue sample is derived from a needle biopsy sample, a fine-needle aspirate, a cytology specimen, or a bone decalcification, or
wherein positive staining is determined using a bright-field light microscope, a microscope objective, a computer monitor and imaging software, or a combination thereof, optionally, wherein the imaging software comprises whole slide imaging software.

20. (canceled)

21. (canceled)

22. A method for diagnosing a tumor or a cancer by determining if a tissue sample is positive for expression of Claudin 18.2, comprising: determining a Claudin 18.2 interpretive status in a tissue sample by a method of claim 1, wherein a Claudin 18.2 score of about 10% or greater of neoplastic or cancer cells having Claudin 18.2 cellular membrane staining at an intensity of 1+ or greater is positive, optionally, wherein the tumor is a gastric cancer, a stomach cancer, a gastric cancer metastases, a gastroesophageal junction (GEJ) cancer, an esophageal cancer, a colorectal cancer, colitis associated colorectal adenocarcinoma, a colon cancer, a non-squamous non-small cell lung cancer (nsNSCLC), a non-small cell lung cancer (NSCLC), an adenocarcinoma, Cholangiocarcinoma, biliary intraepithelial neoplasia, Pancreatic cancer, pancreatic adenocarcinoma, pancreatic cancer metastases, pancreaticobiliary adenocarcinoma, Genitourinary tract cancer, biliary carcinoma, and biliary tract cancer.

23. (canceled)

24. A method for treating or ameliorating a tumor or a cancer in a patient, comprising determining a Claudin 18.2 interpretive status in a tissue sample from the patient using a method of claim 1, and treating the patient with a cancer therapeutic to which the patient is likely to respond favorably based on the Claudin 18.2 interpretive status, optionally wherein the tissue sample has a Claudin 18.2 score of about 20% or greater of neoplastic or cancer cells having Claudin 18.2 cellular membrane staining at an intensity of 2+ or greater, and optionally, wherein the tumor or cancer is a gastric cancer, a stomach cancer, a gastric cancer metastases, a gastroesophageal junction (GED) cancer, an esophageal cancer, a colorectal cancer, colitis associated colorectal adenocarcinoma, a colon cancer, a non-squamous non-small cell lung cancer (nsNSCLC), a non-small cell lung cancer (NSCLC), an adenocarcinoma, Cholangiocarcinoma, biliary intraepithelial neoplasia, Pancreatic cancer, pancreatic adenocarcinoma, pancreatic cancer metastases, pancreaticobiliary adenocarcinoma, Genitourinary tract cancer, biliary carcinoma, or biliary tract cancer.

25. (canceled)

26. (canceled)

27. The method of claim 24, wherein the cancer therapeutic comprises administration to the patient an anti-cancer drug or an anti-cancer therapy, optionally comprising an antibody drug conjugate, a small molecule therapy, an immunotherapy, a monoclonal antibody therapy, an adoptive cell therapy, a T-cell receptor therapy, or a chimeric antigen receptor (CAR) T-cell therapy.

28. (canceled)

29. A kit comprising an antibody which specifically binds to Claudin 18.2 and Claudin 18.2 scoring guidelines as set forth in a method of claim 1.

30. A method for assessing the extent of Claudin 18.2 expression in a tissue sample from an individual comprising: contacting the tissue sample or a portion thereof comprising cancer or neoplastic cells with an antibody or a portion thereof which specifically binds to Claudin 18.2; determining a total number of viable neoplastic or cancer cells having Claudin 18.2 cellular membrane staining; determining a total number of staining and non-staining viable neoplastic or cancer cells; and determining a Claudin 18.2 score (%) by dividing the number of Claudin 18.2 staining viable neoplastic or cancer cells in the sample or portion thereof that are specifically bound by the antibody by the total number of staining and non-staining viable cancer or neoplastic cells and multiplying the result by 100, thereby obtaining the Claudin 18.2 score (%).


SEQUENCE LISTING (1 sequence)

SEQ ID NO: 1 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV